(19) United States
(12) Patent Application Publication
(10) Pub. No.: US 2021/0045807 A1
(43) Pub. Date: Feb. 18, 2021

(54) HAIR REMOVAL DEVICE AND METHOD BASED ON LED LIGHT SOURCE

(71) Applicant: Shenzhen Fansizhe Science And Technology Co., Ltd., Shenzhen (CN)

(72) Inventor: Endang CHENG, Shenzhen (CN)

(73) Assignee: Shenzhen Fansizhe Science And Technology Co., Ltd.

(21) Appl. No.: 16/816,061

(22) Filed: Mar. 11, 2020

(30) Foreign Application Priority Data
Aug. 15, 2019 (CN) 201910755732.4

(51) Int. Cl. A61B 18/18; H05B 45/18

(52) U.S. Cl. CPC A61B 18/18; A61B 2018/00476; H05B 45/18

(57) ABSTRACT

A hair removal device based on an LED light source comprises a shell provided with an LED hair removal lamp panel. A circuit board is arranged in the shell and is provided with a hair removal driving circuit, which is electrically connected to the LED hair removal lamp panel and a main control circuit. The LED hair removal lamp panel is preferably used as a light source, fulfills more uniform light distribution, and can be controlled by the main control circuit to emit light waves with the corresponding wavelength to realize hair removal. The hair removal device can control the light wavelength more easily, improves the hair removal effect, and greatly reduces potential safety hazards.

### BACKGROUND OF THE PRESENT INVENTION

### Field of Invention

The invention relates to hair removal devices, in particular to a hair removal device and method based on an LED light source.

### Description of Related Arts

Hair removal devices are small devices used by people to remove hair. Existing hair removal devices typically adopt optical hair removal which is realized by controlling the light intensity of a lamp tube arranged on an operation head of the hair removal devices. However, due to the difficulty in controlling the light intensity and concentrated light sources, poor hair removal effects or skin scalds often occur in actual use, thus causing potential safety hazards and resulting in poor user experience. Thus, the product's effects cannot meet market and user demands.

### SUMMARY OF THE PRESENT INVENTION

The technical issue to be settled by the invention is to overcome the defects of the prior art by providing a hair removal device and method based on an LED light source. The hair removal device and method fulfills uniform light distribution and easy control over light wavelengths, thus improving the hair removal effect and reducing potential safety hazards.

The technical solution adopted by the invention to settle the above-mentioned technical issue is as follows:

A hair removal device based on an LED light source comprises a shell provided with an LED hair removal lamp panel, wherein a circuit board is arranged in the shell and is provided with a hair removal driving circuit electrically connected to the LED hair removal lamp panel and used for driving the LED hair removal lamp panel to emit light, as well as a main control circuit electrically connected to the hair removal driving circuit and used for sending a control instruction to the hair removal driving circuit which in turn drives the LED hair removal lamp panel to emit light waves with a preset wavelength to remove hair.

Preferably, the main control circuit is used for sending a control instruction with a preset duty ratio to the hair removal driving circuit which in turn drives the LED hair removal lamp panel according to the corresponding control instruction to emit light waves with a preset wavelength of 750 nm or 940 nm.

Preferably, the LED hair removal lamp panel includes a plurality of lamp beads distributed in an array manner.

Preferably, the circuit board is provided with an operating mode selection circuit electrically connected to the main control circuit and used for keying a selection instruction to the main control circuit, as well as a power circuit used for supplying power.

Preferably, a fan and a radiator are arranged in the shell, the LED hair removal lamp panel is fixed to the radiator, and the fan is arranged adjacent to the radiator so as to cool the radiator.

Preferably, the circuit board is provided with a fan driving circuit electrically connected between the main control circuit and the fan and used for executing a driving instruction output by the main control circuit to drive the fan to operate, as well as a fan abnormality detection circuit electrically connected between the fan driving circuit and the main control circuit and used for acquiring a signal output by the fan driving circuit and feeding a fan abnormality signal back to the main control circuit when the fan driving circuit fails to drive the fan to operate.

Preferably, the LED hair removal lamp panel is provided with an NTC thermistor used for acquiring a temperature signal of the LED hair removal lamp panel, and the circuit board is provided with a hair removal over-temperature detection circuit which is electrically connected between the NTC thermistor and the main control circuit and used for receiving temperature data acquired by the NTC thermistor and feeding a hair removal over-temperature alarm signal back to the main control circuit when the temperature data reaches a preset value.

Preferably, the circuit board is provided with a hair removal abnormality detection circuit which is electrically connected between the LED hair removal lamp panel and the main control circuit and used for feeding a hair removal abnormality alarm signal back to the main control circuit when a driving signal accessed to the LED hair removal lamp panel is abnormal.

A hair removal method based on an LED light source is implemented on the basis of a hair removal device comprising a shell provided with an LED hair removal lamp panel, wherein a circuit board is arranged in the shell and provided with a hair removal driving circuit electrically connected to the LED hair removal lamp panel and a main control circuit electrically connected to the hair removal driving circuit. The method comprises the following steps: S1, sending, by the main control circuit, a control instruction to the hair removal driving circuit; and S2, driving, by the hair removal driving circuit, the LED hair removal lamp panel according to the control instruction sent from the main control circuit to emit light waves with a preset wavelength to remove hair.

Preferably, in S2, the main control circuit sends a control instruction with a preset duty ratio to the hair removal driving circuit which in turn drives the LED hair removal lamp panel according to the corresponding control instruction to emit light waves with a wavelength of 750 nm or 940 nm.

In the present disclosure, the main control circuit sends the control instruction to the LED hair removal lamp panel which is driven according to the control instruction sent from the main control circuit to emit the light waves with the preset wavelength to remove hair. The LED hair removal lamp panel is preferably used as a light source of the hair removal device, fulfills more uniform light distribution, can be controlled by the corresponding control instruction from the main control circuit to emit the light waves with the corresponding wavelength to realize hair removal. Compared with the prior art, the hair removal device can control the light wavelength more easily, improves the hair removal effect, and greatly reduces potential safety hazards, thus better meeting user and market demands.

### BRIEF DESCRIPTION OF THE DRAWINGS

FIG. 1 is a circuit block diagram of a hair removal device of the invention;

FIG. 2 is a circuit diagram of an LED hair removal lamp panel;

FIG. 3 is a first partial circuit diagram of the hair removal device of the invention;

FIG. 4 is a second partial circuit diagram of the hair removal device of the invention;

FIG. 5 is a third partial circuit diagram of the hair removal device of the invention;

FIG. 6 is a first perspective view of the hair removal device in a preferred embodiment of the invention;

FIG. 7 is a second perspective view of the hair removal device in the preferred embodiment of the invention;

FIG. 8 is an exploded view of the hair removal device in the preferred embodiment of the invention.

### DETAILED DESCRIPTION OF THE PREFERRED EMBODIMENT

The invention is further expounded below in combination with the accompanying drawings and embodiments.

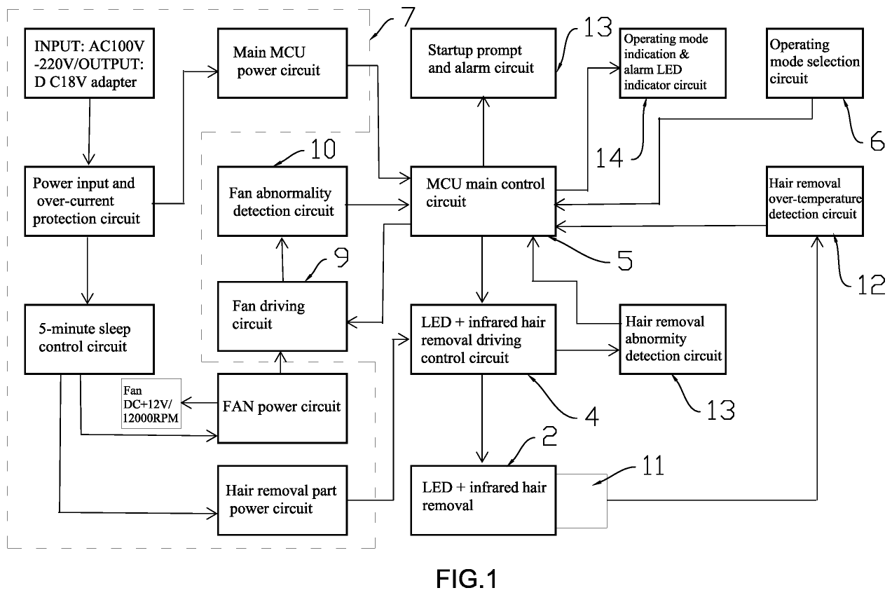

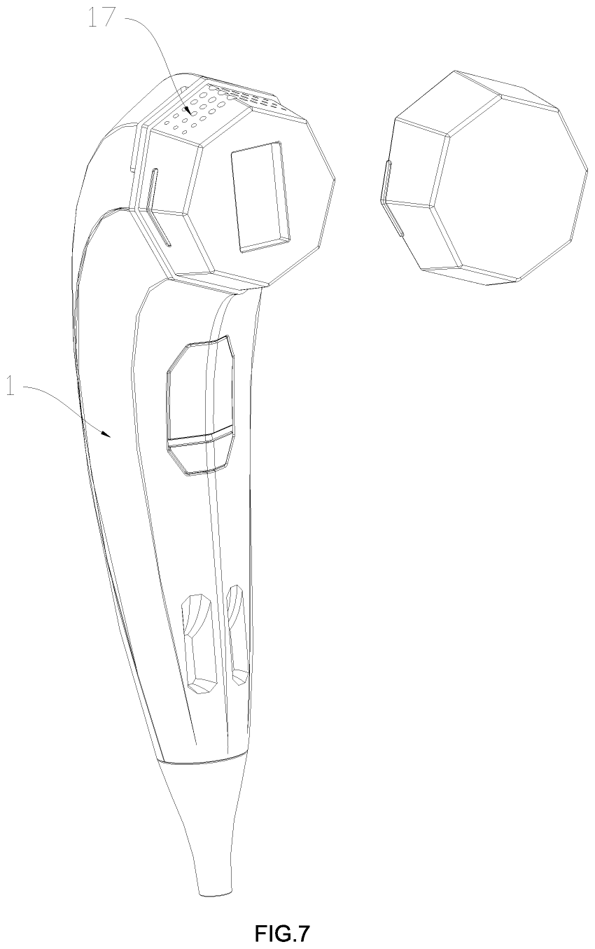

The invention discloses a hair removal device based on an LED light source. As shown in FIG. 1 to FIG. 8, the hair removal device comprises a shell 1 provided with an LED hair removal lamp panel 2, wherein a circuit board 3 is arranged in the shell 1 and is provided with:

a hair removal driving circuit 4 electrically connected to the LED hair removal lamp panel 2 and used for driving the LED hair removal lamp panel 2 to emit light; and

a main control circuit 5 electrically connected to the hair removal driving circuit 4 and used for sending a control instruction to the hair removal driving circuit 4 which in turn drives the LED hair removal lamp panel 2 to emit light waves with a preset wavelength to remove hair.

In the hair removal device, the main control circuit 5 sends the control instruction to the hair removal driving circuit 4 which in turn drives the LED hair removal lamp panel 2 to emit the light waves with the preset wavelength light to removal hair according to the control instruction sent from the main control circuit 5. The LED hair removal lamp panel 2 is preferably used as a light source of the hair removal device, fulfills more uniform light distribution, and can be driven according to the corresponding control instruction from the main control circuit 5 to emit light waves with the corresponding wavelength to realize hair removal. Compared with the prior art, the hair removal device can control the light wavelength more easily, improves the hair removal effect, and greatly reduces potential safety hazards, thus bettering meeting users and market demands.

Wherein, LED light waves convert electric energy into light energy by means of high-intensity radiation and releases the light energy which penetrates through the epidermis to reach the dermis to selectively act on hair shaft melanocytes and hair follicle melanocytes, and melanin accounting for the majority of growing hair follicle cells is extremely sensitive to photons and will be destroyed after hair follicles absorb the energy, so that the whole hair falls off.

Preferably, the main control circuit 5 is used for sending the control instruction of a preset duty ratio to the hair removal driving circuit 4 which in turn drives the LED hair removal lamp panel 2 according to the corresponding control instruction to emit light waves with a wavelength of 750 nm or 940 nm. The hair removal device works under the light irradiation of 750 nm and 940 nm, and according to actual tests, hair growth is effectively suppressed under the light irradiation of 750 nm and 940 nm according to light selectivity, so that the hair removal effect is achieved

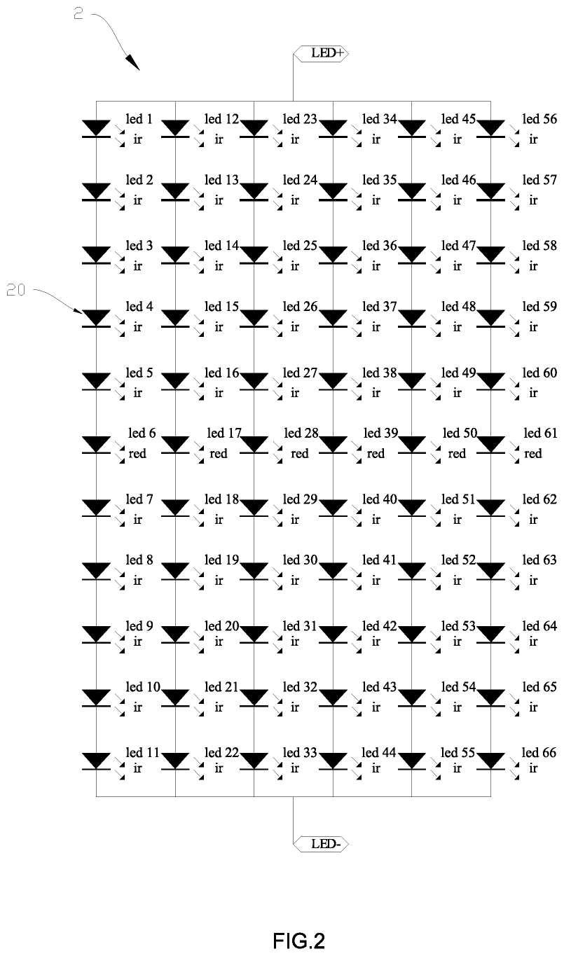

Particularly, in this embodiment, referring to FIG. 2, the LED hair removal lamp panel 2 includes a plurality of lamp beads 20 distributed in an array manner.

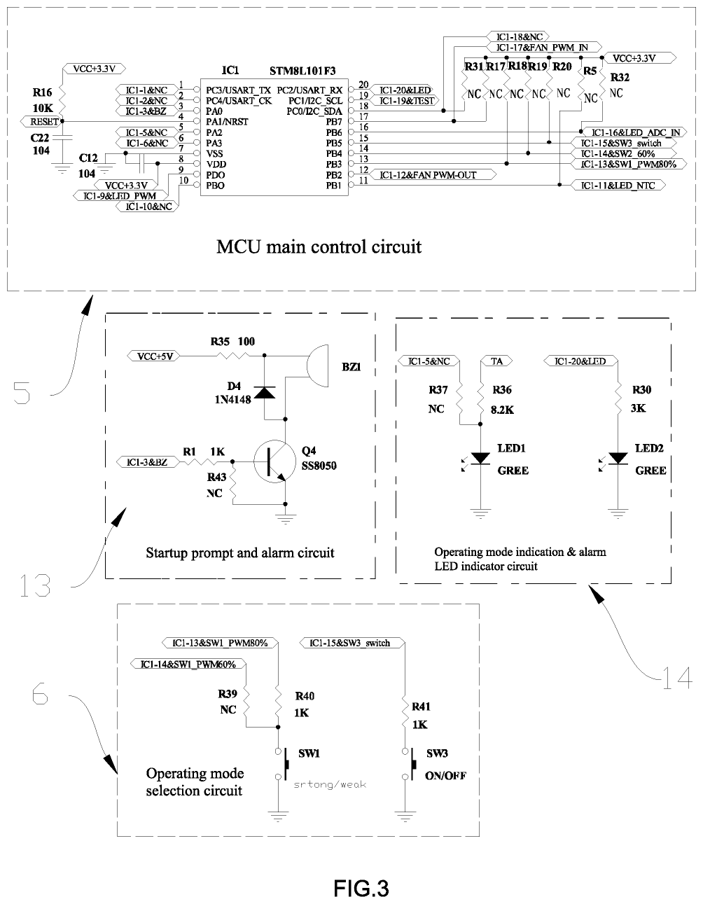

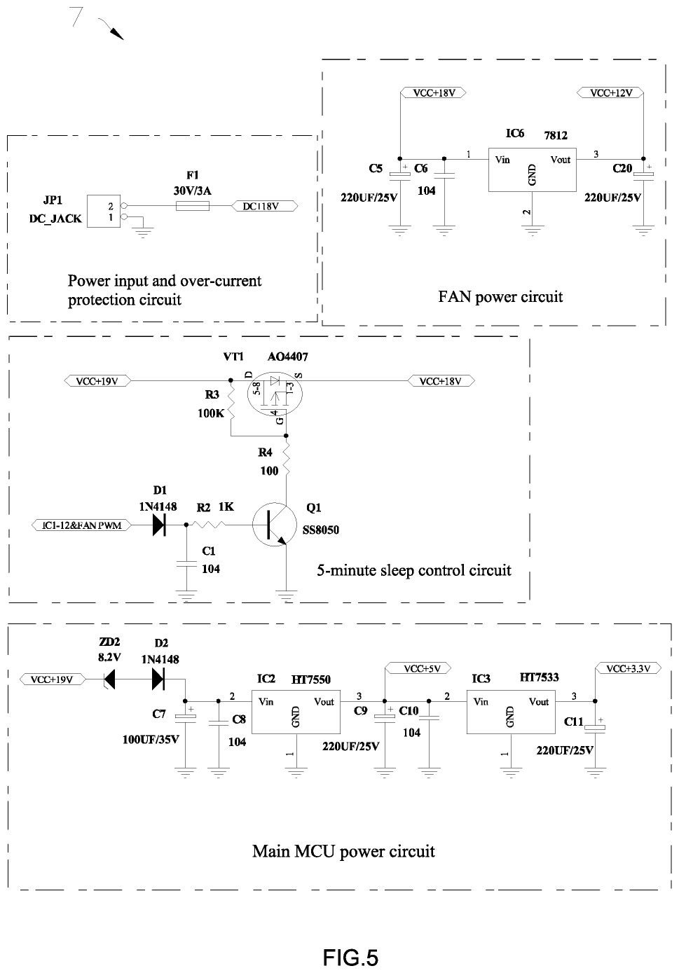

In this embodiment, as shown in FIG. 1, FIG. 3, and FIG. 5, the circuit board 3 is provided with an operating mode selection circuit 6 electrically connected to the main control circuit 5 and used for keying a selection instruction to the main control circuit 5, as well as a power circuit 7 used for supplying power, wherein the operating mode selection circuit 6 is composed of an appropriate number of keys, and operating modes involved are as follows:

1. In the standby mode, when an ON/OFF (mode switching) key is long pressed by hand for 2 seconds, a buzzer gives two “BB” sounds, a host enters into the operating mode, and a fan continuously rotates; and when the ON/OFF (switching) key is long pressed by hand for 2 seconds again, the device is turned off, and the fan stops after rotating for a preset time.

2. In the operating mode, a the lamp holder of the hair removal device is put close to the skin, then an activate key is pressed by hand to make the device enter into a low-gear (60% duty ratio) hair removal mode; in this mode, a hair removal LED lights up, and a hair removal indicator LED flickers at a low gear (60% duty ratio); after the activate key is released by hand, the hair removal LED stops lighting, and the hair removal indicator LED is turned off; and after the activate key is pressed again, hair removal is carried out again.

3. In the low-gear (60% duty ratio) hair removal mode, when the ON/OFF (mode switching) key is short pressed, the device is switched to a high-gear (80% duty ratio) hair removal mode; and when the activate key is pressed by hand, the device enters into the high-gear (80% duty ratio) hair removal mode.

4. In the operating mode, when the ON/OFF (mode switching) key is short pressed, the device can be selected to the high-gear (80% duty ratio) hair removal mode or the low-gear (60% duty ratio) hair removal mode.

5. If the key is not pressed within 5 minutes, the device enters into the a sleep mode.

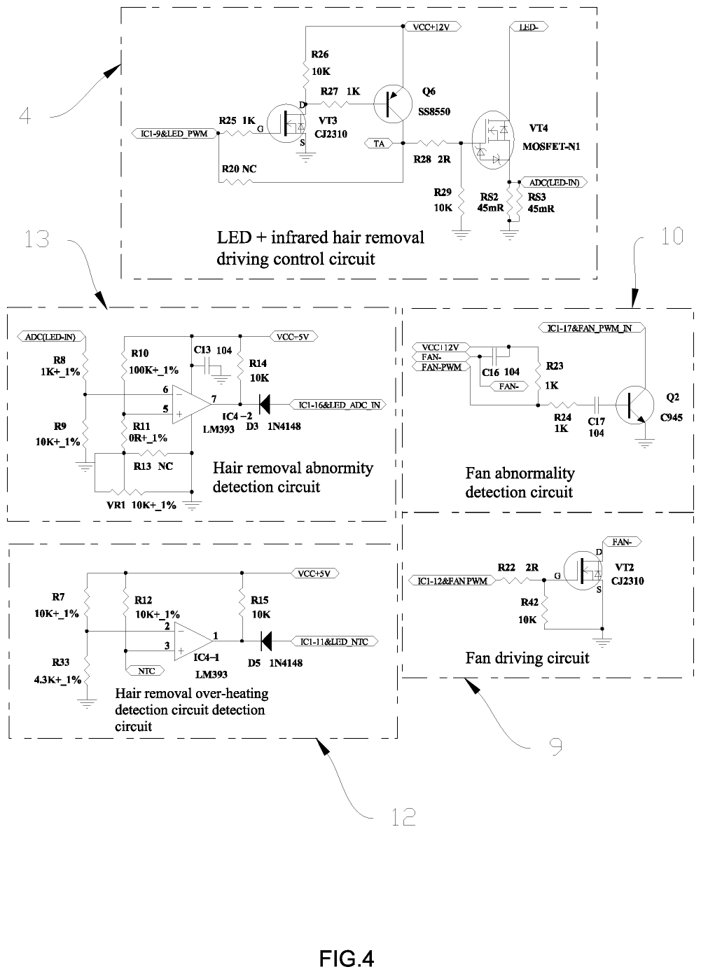

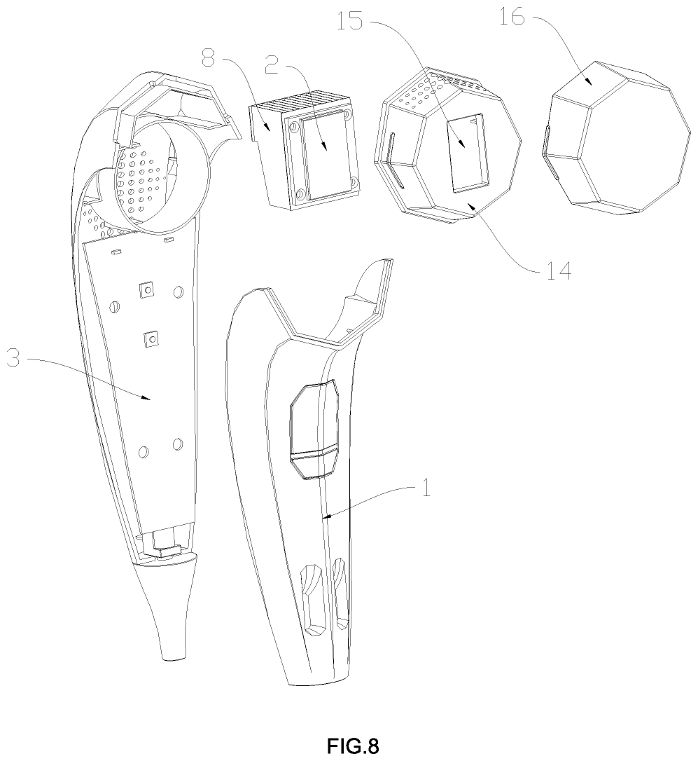

In this embodiment, as shown in FIG. 1, FIG. 3, FIG. 4, and FIG. 8, the fan (unmarked) and a radiator 8 are arranged in the shell 1 to fulfill cooling in time, the LED hair removal lamp panel 2 is fixed to the radiator 8, and the fan is arranged adjacent to the radiator 8 so as to cool the radiator 8.

In order to make sure that the hair removal device has a good cooling effect and to prevent an operation head against overt-temperature, in this embodiment, the circuit board 3 is provided with:

a fan driving circuit 9 electrically connected between the main control circuit 5 and the fan and used for executing a driving instruction output by the main control circuit 5 to drive the fan to operate; and

a fan abnormality detection circuit 10 electrically connected between the fan driving circuit 9 and the main control circuit 5 and used for acquiring a signal output by the fan driving circuit 9 and feeding a fan abnormality signal back to the main control circuit 5 when the fan driving circuit 9 fails to drive the fan to operate.

Preferably, in this embodiment, temperature detection and alarm prompt functions are further fulfilled. Particularly, the LED hair removal lamp panel 2 is provided with an NTC thermistor 11 used for acquiring a temperature signal of the LED hair removal lamp panel 2; and

The circuit board 3 is provided with a hair removal over-temperature detection circuit 12 which is electrically connected between the NTC thermistor 11 and the main control circuit 5 and used for receiving temperature data acquired by the NTC thermistor 11 and feeding a hair removal over-temperature alarm signal back to the main control signal 5 when the temperature data reaches a preset value.

In this embodiment, the circuit board 3 is provided with a hair removal abnormality detection circuit 13 which is electrically connected between the LED hair removal lamp panel 2 and the main control circuit 5 and used for feeding a hair removal abnormality alarm signal back to the main control circuit 5 when a driving signal accessed to the LED hair removal lamp panel 2 is abnormal.

In order to better describe the technical solution of the invention, the invention further relates to a hair removal method based on an LED light source. As shown in FIG. 1 to FIG. 8, the hair removal method based on an LED light source is implemented on the basis of a hair removal device comprising a shell 1, wherein the shell 1 is provided with an LED hair removal lamp panel 2, a circuit board 3 is arranged in the shell 1 and provided with a hair removal driving circuit 4 and a main control circuit 5 which are electrically connected to the LED hair removal lamp panel 2. The method comprises the following steps:

S1, the main control circuit 5 sends a control instruction to the LED hair removal lamp panel 2; and

S2, the LED hair removal lamp panel 2 is driven according to the control instruction sent from the main control circuit 5 to emit light waves with a preset wavelength light to remove hair.

In S2 of the method, the main control circuit 5 sends the control instruction with a preset duty ratio to the LED hair removal lamp panel 2 which is in turn driven according to the corresponding control instruction to send light waves with a wavelength of 750 nm or 940 nm.

As an auxiliary circuit of the invention, as shown in FIG. 1 and FIG. 4, the circuit board 3 is provided with:

A startup prompt and alarm circuit 130 electrically connected to the main control circuit 5 and used for giving an alarm prompt when the hair removal device starts; and

An operating mode indication and alarm unit 14 electrically connected to the main control circuit 5 and used for giving sound and light prompts.

The circuit gives the alarm prompt when the hair removal device starts to alert users that the LED hair removal lamp panel 2 can emit light waves with certain energy at any time.

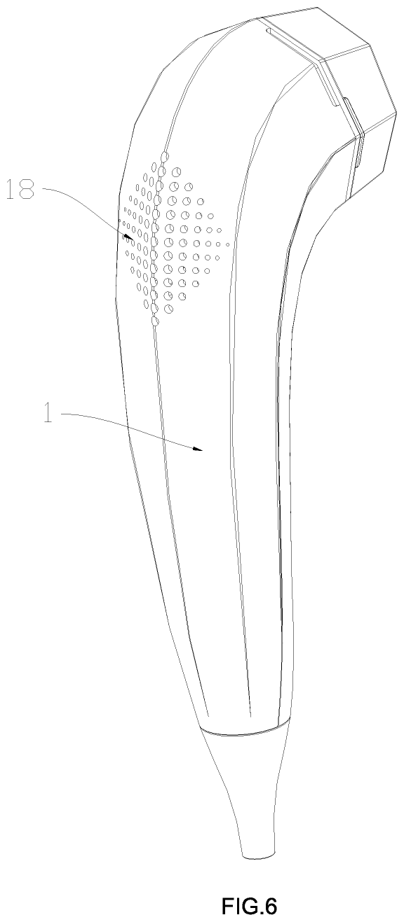

Preferably, as shown in FIG. 6 to FIG. 8, the shell 1 is in the shape of a handle, and an operation head 14 is arranged at the end of the shell 1 and is provided with a window and glass 15 covering the window and aligned with the LED hair removal lamp panel 2. The above structure can be conveniently held by the users and enables light to be uniformly scattered via the glass 15.

In order to achieve a protection effect, in this embodiment, the operation head 14 is covered with a protective cover 16 which is detachably connected to the operation head 14 in a matched manner.

In this embodiment, a cooling channel is arranged in the shell 1 to improve the cooling effect. Particularly, air inlets 17 are formed in the operation head 14, air outlets 18 are formed in the back side of the shell 1, and a fan is located between the air inlets 17 and the air outlets 18.

Particularly, as shown in FIG. 1 to FIG. 5, in this embodiment, the main control circuit 5 includes a single-chip microcomputer ICI and a peripheral circuit. Furthermore, the LED hair removal lamp panel 2 includes infrared lamp beads capable of emitting infrared rays.

Particularly, in this embodiment, the hair removal driving circuit 4 includes a PNP transistor Q6, an MOS transistor VT3, and an MOS transistor VT4, wherein the MOS transistor VT3 has a gate connected to the main control circuit 5 through a resistor R25, a drain connected to a 12V power terminal through a resistor R26 and to a base of the PNP transistor Q6 through a resistor R27, and a source grounded, the PNP transistor Q6 has an emitter connected to the 12V power terminal and a collector connected to a gate of the MOS transistor VT4 through a resistor R28, the MOS transistor VT4 has a source grounded and a drain connected to a cathode of the LED hair removal lamp panel 2, and an anode of the LED hair removal panel 2 is connected to a high potential.

In the above circuit, the main control circuit 5 outputs a PWM pulse driving signal to the gate of the MOS transistor VT3 through the resistor R25, then the MOS transistor VT3 is turned on and lowers a base voltage of the PNP transistor Q6, and afterwards, the PNP transistor Q6 is turned on and drives the MOS transistor VT4 to be turned on, so that a power-on circuit of the LED hair removal lamp panel 2 is turned on, and the LED hair removal lamp panel 2 starts to operate. In actual application, the main control circuit 5 loads a PWM pulse signal with the corresponding duty ratio to the gate of the MOS transistor VT3 to control and adjust the LED hair removal lamp panel 2.

In order to fulfill hair removal abnormality monitoring, in this embodiment, a hair removal abnormality detection circuit 13 includes a comparator IC4-2, wherein sampling resistors (RS2 and RS3) are connected in series between the source of the MOS transistor VT4 and the ground, a voltage signal from the source of the MOS transistor VT4 is divided by a resistor R8 and a resistor R9 and is then transmitted to a reverse terminal of the comparator IC4-2, a reference voltage obtained after division by a resistor R10 and a resistor R11 is accessed to an in-phase terminal of the comparator IC4-2 is used for accessing, and the comparator IC4-2 is used for comparing a voltage of the in-phase terminal with a voltage of the reverse terminal and feeding a hair removal abnormality alarm signal back to the main control circuit 5 when the MOS transistor VT4 is abnormal and the driving signal accessed to the LED hair removal lamp panel 2 is determined to be abnormal.

In the above circuit, when a current of the MOS transistor VT4 is abnormally increased, the LED hair removal lamp panel 2 will be in an abnormal over-current state; and at this moment, the voltage of the reverse terminal of the comparator IC4-2 is higher than that of the in-phase terminal of the comparator IC4-2, a high level at an output terminal of the comparator IC4-2 turns into a low level, and the hair removal abnormality alarm signal is output to the main control circuit 5.

In order to fulfill accurate temperature monitoring, in this embodiment, a hair removal over-temperature detection circuit 12 includes a comparator IC4-1, wherein a voltage signal acquired by an NTC thermistor 11 is transmitted to an in-phase terminal of the comparator IC4-1, a reference voltage obtained after division by a resistor R7 and a resistor R33 is accessed to a reverse terminal of the comparator IC4-1, and the comparator IC4-1 is used for feeding a hair removal over-temperature alarm signal back to the main control circuit 5 when the voltage signal acquired by the NTC thermistor 11 reaches a preset reference voltage value.

In this embodiment, the comparator IC4-1 is preferably used as a main part for temperature monitoring and judgment, and such a temperature monitoring manner is conductive to the setting of a temperature threshold. The comparator IC4-1 is easy to design and modulate.

The above embodiments are only preferred ones of the invention, and are not intended to limit the invention. All modifications, equivalent substitutes, or improvements made within the technical scope of the invention should also fall within the protection scope of the invention.

What is claimed is:

1. A hair removal device based on an LED light source, comprising a shell (1) provided with an LED hair removal lamp panel (2), wherein a circuit board (3) is arranged in the shell (1) and is provided with:
a hair removal driving circuit (4) electrically connected to the LED hair removal lamp panel (2) and used for driving the LED hair removal lamp panel (2) to emit light; and
a main control circuit electrically connected to the hair removal driving circuit (4) and used for sending a control instruction to hair removal driving circuit (4) which in turn drives the LED hair removal lamp panel (2) to emit light waves with a preset wavelength to remove hair.

2. The hair removal device based on an LED light source according to claim 1, wherein the main control circuit (5) is used for sending a control instruction with a preset duty ratio to the hair removal driving circuit (4) which in turn drives the LED hair removal lamp panel (2) according to the corresponding control instruction to emit light waves with a wavelength of 750 nm or 940 nm.

3. The hair removal device based on an LED light source according to claim 1, wherein the LED hair removal lamp panel (2) includes a plurality of lamp beads (20) distributed in an array manner.

4. The hair removal device based on an LED light source according to claim 1, wherein the circuit board (3) is provided with an operating mode selection circuit (6) electrically connected to the main control circuit (5) and used for keying a selection instruction to the main control circuit (5), as well as a power circuit (7) used for supplying power.

5. The hair removal device based on an LED light source according to claim 1, wherein a fan and a radiator (8) are arranged in the shell (1), the LED hair removal lamp panel (2) is fixed to the radiator (8), and the fan is arranged adjacent to the radiator (8) so as to cool the radiator (8).

6. The hair removal device based on an LED light source according to claim 5, wherein the circuit board (3) is provided with:
a fan driving circuit (9) electrically connected between the main control circuit (5) and the fan and used for executing a driving instruction output by the main control circuit (5) to drive the fan to operate; and
a fan abnormality detection circuit (10) electrically connected between the fan driving circuit (9) and the main control circuit (5) and used for acquiring a signal output by the fan driving circuit (9) and feeding a fan abnormality signal back to the main control circuit (5) when the fan driving circuit (9) fails to drive the fan to operate.

7. The hair removal device based on an LED light source according to claim 1, wherein the LED hair removal lamp panel (2) is provided with an NTC thermistor (11) used for acquiring a temperature signal of the LED hair removal lamp panel (2); and
the circuit board (3) is provided with a hair removal over-temperature detection circuit which (12) is electrically connected between the NTC thermistor (11) and the main control circuit (5) and used for receiving temperature data acquired by the NTC thermistor (11) and feeding a hair removal over-temperature alarm signal back to the main control circuit (5) when the temperature data reaches a preset value.

8. The hair removal device based on an LED light source according to claim 1, wherein the circuit board (3) is provided with a hair removal abnormity detection circuit (13) which is electrically connected between the LED hair removal lamp panel (2) and the main control circuit (5) and used for feeding a hair removal abnormity alarm signal back to the main control circuit (5) when a driving signal accessed to the LED hair removal lamp panel (2) is abnormal.

9. A hair removal method based on an LED light source, being implemented on the basis of a hair removal device comprising a shell (1) provided with an LED hair removal lamp panel (2), a circuit board (3) being arranged in the shell (1) and provided with a hair removal driving circuit (4) electrically connected to the LED hair removal lamp panel (2) and a main control circuit (5) electrically connected to the hair removal driving circuit (4), and the method comprising the following steps:
S1, sending, by the main control circuit (5), a control instruction to the hair removal driving circuit (4); and
S2, driving, by the hair removal driving circuit (4), the LED hair removal lamp panel (2) according to the control instruction sent from the main control circuit (5) to emit light waves with a preset wavelength to remove hair.

10. The hair removal method based on an LED light source according to claim 9, wherein in S2, the main control circuit (5) sends a control instruction with a preset duty ratio to the hair removal driving circuit (4) which in turn drives the LED hair removal lamp panel (2) according to the corresponding control instruction to emit light waves with a wavelength of 750 nm or 940 nm.
